SEQUENCE LISTING (4 sequences)

SEQ ID NO: 1 - protein, 423 aa, Homo sapiens
MVPHLLLLCLLPLVRATEPHEGRADEQSAEAALAVPNASHFFSWNNYTFSDWQNFVGRRRYGAESQNPTVKALLIVAYSFIIVFSLFGNVLVCHVIFKNQRMHSATSLFIVNLAVADIMITLLNTPFTLVRFVNSTWIFGKGMCHVSRFAQYCSLHVSALTLTAIAVDRHQVIMHPLKPRISITKGVIYIAVIWTMATFFSLPHAICQKLFTFKYSEDIVRSLCLPDFPEPADLFWKYLDLATFILLYILPLLIISVAYARVAKKLWLCNMIGDVTTEQYFALRRKKKKTIKMLMLVVVLFALCWFPLNCYVLLLSSKVIRTNNALYFAFHWFAMSSTCYNPFIYCWLNENFRIELKALLSMCQRPPKPQEDRPPSPVPSFRVAWTEKNDGQRAPLANNLLPTSQLQSGKTDLSSVEPIVTMS

SEQ ID NO: 2 - DNA, 1272 bp, Homo sapiens
atggtccctcacctcttgctgctctgtctcctccccttggtgcgagccaccgagccccacgagggccgggccgacgagcagagcgcggaggcggccctggccgtgcccaatgcctcgcacttcttctcttggaacaactacaccttctccgactggcagaactttgtgggcaggaggcgctacggcgctgagtcccagaaccccacggtgaaagccctgctcattgtggcttactccttcatcattgtcttctcactctttggcaacgtcctggtctgtcatgtcatcttcaagaaccagcgaatgcactcggccaccagcctcttcatcgtcaacctggcagttgccgacataatgatcacgctgctcaacacccccttcactttggttcgctttgtgaacagcacatggatatttgggaagggcatgtgccatgtcagccgctttgcccagtactgctcactgcacgtctcagcactgacactgacagccattgcggtggatcgccaccaggtcatcatgcaccccttgaaaccccggatctcaatcacaaagggtgtcatctacatcgctgtcatctggaccatggctacgttcttttcactcccacatgctatctgccagaaattatttaccttcaaatacagtgaggacattgtgcgctccctctgcctgccagacttccctgagccagctgacctcttctggaagtacctggacttggccaccttcatcctgctctacatcctgcccctcctcatcatctctgtggcctacgctcgtgtggccaagaaactgtggctgtgtaatatgattggcgatgtgaccacagagcagtactttgccctgcggcgcaaaaagaagaagaccatcaagatgttgatgctggtggtagtcctctttgccctctgctggttccccctcaactgctacgtcctcctcctgtccagcaaggtcatccgcaccaacaatgccctctactttgccttccactggtttgccatgagcagcacctgctataaccccttcatatactgctggctgaacgagaacttcaggattgagctaaaggcattactgagcatgtgtcaaagacctcccaagcctcaggaggacaggccaccctccccagttccttccttcagggtggcctggacagagaagaatgatggccagagggctccccttgccaataacctcctgcccacctcccaactccagtctgggaagacagacctgtcatctgtggaacccattgtgacgatgagttag

SEQ ID NO: 3 - protein, 423 aa, mouse
MKVPPVLLLFLLSSVRATEQPQVVTEHPSMEAALTGPNASSHFWANYTFSDWQNFVGRRRYGAESQNPTVKALLIVAYSFTIVFSLFGNVLVCHVIFKNQRMHSATSLFIVNLAVADIMITLLNTPFTLVRFVNSTWVFGKGMCHVSRFAQYCSLHVSALTLTAIAVDRHQVIMHPLKPRISITKGVIYIAVIWVMATFFSLPHAICQKLFTFKYSEDIVRSLCLPDFPEPADLFWKYLDLATFILLYLLPLFIISVAYARVAKKLWLCNTIGDVTTEQYLALRRKKKTTVKMLVLVVVLFALCWFPLNCYVLLLSSKAIHTNNALYFAFHWFAMSSTCYNPFIYCWLNENFRVELKALLSMCQRPPKPQEDRLPSPVPSFRVAWTEKSHGRRAPLPNHHLPSSQIQSGKTDLSSVEPVVAMS

SEQ ID NO: 4 - DNA, 1272 bp, mouse
atgaaggttcctcctgtcctgcttctctttcttctgtcctcagtgcgagctactgagcaaccgcaggtcgtcactgagcatcccagcatggaggcagccctgaccgggcccaacgcctcctcgcacttctgggccaactacactttctctgactggcagaacttcgtgggcaggagacgttatggggccgagtcccagaaccccacggtgaaagcactgctcatcgtggcctactcattcaccatcgtcttctcgctcttcggtaatgtcctggtctgtcatgtcatcttcaagaaccagcgcatgcactcggccaccagcctcttcattgtcaacctggcagtggcggacatcatgatcacattgctcaacacgcccttcactttggtccgctttgtgaacagcacatgggtgtttgggaagggcatgtgtcatgtcagtcgctttgctcagtactgttctctacatgtctcagcactgactctgacagctatcgcagtggaccgccaccaggtcatcatgcatccactgaagcctcggatctccatcaccaagggtgtcatatatattgctgtcatctgggtcatggctaccttcttctctctgccacatgccatctgccagaaactgtttaccttcaagtacagtgaggacattgtgcgctccctctgcctgccggacttcccggagccagctgacctcttctggaagtatctggacctggccaccttcatcctgctctacctacttccactcttcattatctcagtggcctatgctcgtgtggccaagaagctgtggctctgtaacaccattggcgacgtgaccacagagcagtacctcgccctgcgacgcaagaagaagaccaccgtgaagatgctggtgcttgtggtagtcctctttgccctctgctggttccctctcaactgctatgtcctcctcttgtccagcaaggccatccacaccaacaatgccctctactttgccttccactggtttgccatgagcagtacttgttataaccccttcatctactgctggctcaatgagaactttagggttgagcttaaggcattgctgagcatgtgccaaaggccacccaagccgcaggaagacaggctaccctccccagttccttccttcagggtggcatggacagagaagagccatggtcggagggctccactacctaatcaccacttgccctcttcccagatccagtctgggaagacagatctgtcatctgtggaacccgttgtggccatgagttag

(19) United States
(12) Patent Application Publication
(10) Pub. No.: US 2003/0129664 A1
(43) Pub. Date: Jul. 10, 2003

(54) Novel Assays

(76) Inventors: Sultan Ahmad, St Laurent (CA); Paola Lembo, St Laurent (CA); Phillippe Walker, St Laurent (CA)

Correspondence Address: WHITE & CASE LLP, PATENT DEPARTMENT, 1155 AVENUE OF THE AMERICAS, NEW YORK, NY, 10036, US

(21) Appl. No.: 10/168,262

(22) PCT Filed: Dec. 15, 2000
(86) PCT No.: PCT/SE00/02563

(30) Foreign Application Priority Data
Dec. 17, 1999 (SE) 9904660-9

(51) Int. Cl. G01N 33/53; G01N 33/567

(52) U.S. Cl. 435/7.2

(57) ABSTRACT

The present invention is directed to assays that can be used to screen for compounds that act as agonists or antagonists of sphingosine-1 PO₄. The assays are based upon the binding of sphingosine-1 PO₄ to the RP-23 receptor.

### FIELD OF THE INVENTION

The present invention is directed to methods for determining whether a test compound modulates interactions between sphingosine-1-PO₄ and a particular G protein-coupled receptor (RP-23). Compounds identified as being effective modulators have potential use as therapeutic agents for treating cardiovascular diseases, inflammatory diseases, and cancer.

### BACKGROUND OF THE INVENTION

A. Sphingosine-1-PO₄

Sphingolipids are located in cell membranes and are composed of three basic components: a polar bead group, an amide-linked fatty acid, and a sphingoid base (long aliphatic chain). The breakdown of sphingolipids produces sphingosine-1-PO₄, a compound involved in cell signaling (Meyer zu Heringdorf et al., FEBS Lett. 410:34-38 (1997)). Although the activities of sphingosine-1-PO₄ are not fully understood, there are reports that suggest that it may serve as an inhibitor of protein kinase C (Hannun et al., Science 243;500-507 (1989)). More recent work suggests that sphingosine-1-PO₄ plays an important role in cell survival, neuronal differentiation, apoptosis, regulation of mitogenesis, hemodynamics, and wound healing. Thus, agents that modulate sphingosine-1-PO₄ activity have potential as therapeutic agents for the treatment of cardiovascular diseases, inflammatory disorders, and cancer.

B. G Protein Coupled Receptors

G protein coupled receptors (GPCRs) constitute a family of proteins sharing a common structural organization characterized by an extracellular N-terminal end, 7 hydrophobic alpha helices putatively constituting transmembrane domains, and an intracellular C-terminal domain. GPCRs bind a wide variety of ligands that trigger intracellular signals through the activation of transducing G proteins (Caron, et al., Rec. Prog. Horm. Res. 48:277-290 (1993); Freedman, et al., Rec. Prog. Horm. Res. 51:319-353 (1996)). More than 300 GPCRs nave been cloned thus far and it is generally assumed that there exist well over 1,000 such receptors. Roughly 50-60% of all clinically relevant drugs act by modulating the functions of various GPCRs (Gudermann, et al., J. Mol. Med. 73:51-63 (1995)).

Among the GPCRs that have been identified and cloned is a gene that encodes RP -23, a protein homologous to the receptors of the tachykinin family. Harrigan, et al., originally described the structure of this receptor and procedures by which it may be obtained from mouse cells (Mol. Endocrinol. 5:1331-1338 (1991)). Methods have also been described for obtaining a corresponding gene from humans (Hinuma, et al., EP 789,076 (1997)). RP-23 is highly expressed in the brain and thymus of animals and, until now, has been considered an orphan receptor.

### SUMMARY OF THE INVENTION

The present invention is based upon the discovery that sphingosine-1 phosphate is a ligand for the RP-23 receptor. Upon binding, sphingosine-1 phosphate causes an increase in receptor-generated adenylyl cyclase activity and an increase in intracellular calcium concentration. Based upon these discoveries, assays have been developed for identifying agents that alter the binding of sphingosine-1 phosphate to RP-23 and resulting cellular activities.

In its first aspect, the invention is directed to a method for determining whether a test compound modulates the binding of sphingosine-1 phosphate to an RP-23 receptor. This is accomplished by performing a first incubation in which a source of RP-23 receptor is incubated with sphingosine-1 phosphate and a test compound. Any form of RP-23 whose amino acid or gene sequence has been described in the literature and which can be synthesized using conventional methods may be used in the assay. However, the preferred receptor is one having the amino acid sequence corresponding to human RP-23 (SEQ ID NO: 1). After incubation is complete, a determination is made of the extent to which sphingosine-1 phosphate has specifically bound to RP-23. This may be accomplished using standard radioimmunoassay or ELISA procedures. The results obtained are then compared with results from a second, control, incubation performed under essentially the same conditions but in which test compound is absent or present at a significantly different concentration. Based upon this comparison, it may be concluded that the test compound modulates sphingosine-1 phosphate binding if the amount of binding observed in the first incubation is either significantly higher or lower than the binding observed in the control incubation. As used herein, the term “significantly” means that the differences observed are statistically significant when analyzed in a manner accepted in the art.

Any source of RP-23 receptor is compatible with the invention provided that the ability to bind to sphingosine-1 phosphate is maintained. Thus, a tissue preparation or preparation of cell membranes may be used. However, cells expressing a recombinant human RP-23 gene (SEQ ID NO: 2) are preferred. In order to quantitate binding, sphingosine-1 phosphate will usually be detectably labeled. For example, a radioactive isotope such as ³²P may be incorporated into its structure. Although it is possible to draw conclusions based upon a single concentration of test compound, it is preferable to perform repeated incubations in which the concentrations of RP-23 and sphingosine-1 phosphate are held constant and the concentration of test compound is varied. If desired, assays may also include a determination of the effect of test compound on the adenylyl cyclase activity of cells or their intracellular calcium concentration.

In a second aspect, the invention is directed to a method for determining the ability of a lest compound to modulate sphingosine-1 phosphate-stimulated RP-23 receptor-mediated activity. Incubations are performed between a cell expressing RP-23, sphingosine-1 phosphate and test compound. The effect of the incubation on intracellular adenylyl cyclase activity and/or intracellular calcium concentration is then determined and compared with the results obtained from control incubations performed under essentially the same conditions but in which test compound is either present at a significantly different concentration or, preferably, absent. It may be concluded that the test compound modulates sphingosine-1 phosphate-stimulated RP-23 receptor-mediated activity if either the adenylyl cyclase activity or intracellular calcium concentration observed is significantly different than that in control incubations. Preferably, the cells used in assays express a recombinant RP-23 gene encoding an amino acid sequence corresponding to human RP-23. In order for the recombinant receptor to bc functional, i.e. to induce adenylyl cyclase activity or an increase in intracellular calcium, it will usually be necessary to co-transfect cells with an appropriate signal transducing G protein such as Gαqi5, a protein in which the C-terminal amino acids of Gαq are changed from EYNLV to DCGLF. This construct allows many Gi-coupled receptors to stimulate phospholipase C (PLC) and may be prepared as described in the literature (Conklin, B. R. et al., Nature 363,274-276 (1993).

Although not essential, the sphingosine-1 phosphate may be detectably labeled and repeated incubations can be performed in which the concentration of receptor and sphingosine-1 phosphate are held constant and the concentration of test compound is varied.

The invention is also directed to a method for determining if a test compound is an antagonist of sphingosine-1 phosphate-mediated RP-23 receptor activation. Unlike the procedures discussed above, this method may be carried out in the absence of the sphingosine-1 phosphate ligand and is based upon the observation that G protein-coupled receptors self-activate when overexpressed. DNA encoding RP-23 is incorporated into an expression vector so that it is operably linked to a promoter The term “operably linked” as used herein means that expression is under the control of the promoter and occurs in such a manner that the receptor made has the correct amino acid sequence. The expression vector is then transfected into a host cell in which its promoter is active. For example, a CMV promoter might be used in combination with human cells or an SV 40 promoter might be used with simian cells. In order to obtain a receptor that induces adenylyl cyclase activity and increased intracellular calcium levels, it will usually also be necessary to overexpress a gene encoding a G protein signal transducer, e.g. Gαqi5, in cells. This may be accomplished by incorporating the G protein DNA sequence into the expression vector containing RP-23 or by co-transfecting the cells with a second expression vector encoding the G protein.

After transfection, cells that have constitutively activated RP-23 receptors may be identified by their having a statistically significant increase in intracellular calcium levels or intracellular adenylyl cyclase activity relative to control cells that have either not undergone transfection or that have been mock transfected. The selected cells are incubated with one or more concentrations of test compound to determine whether this causes a significant decrease in either intracellular adenylyl cyclase activity or intracellular calcium concentration relative to constitutively activated cells not contacted with the test compound. Preferably, this method will be carried out using receptor having the amino acid sequence of human RP-23.

### BRIEF DESCRIPTION OF THE DRAWINGS

FIG. 1: FIG. 1 shows the amino acid sequence of the human RP-23 receptor protein.

FIG. 2: FIG. 2 shows the nucleotide sequence of the human RP-23 gene.

FIG. 3: FIG. 3 shows the amino acid sequence of thc mouse RP-23 receptor protein.

FIG. 4: FIG. 4 shows the nucleotide sequence of the mouse RP-23 gene.

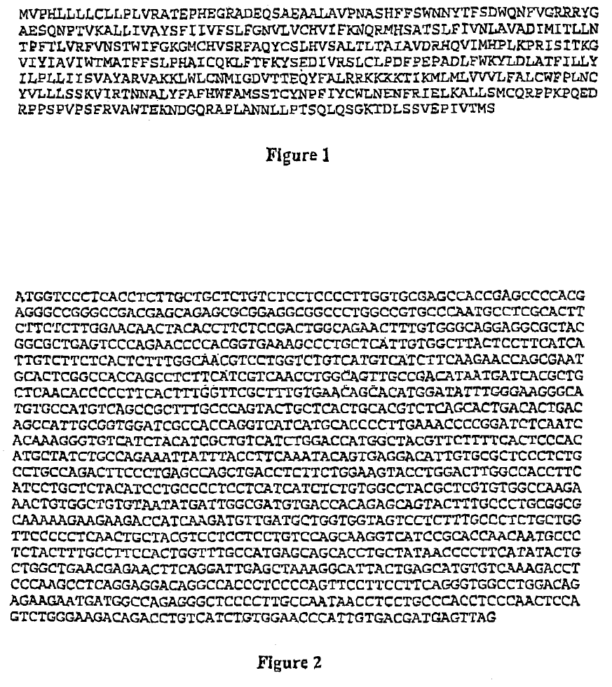

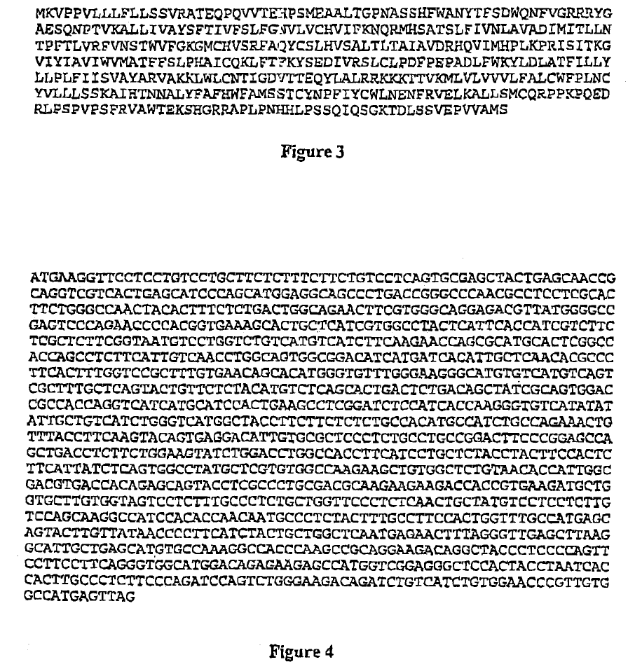

### DEFINITIONS

The description of the invention herein uses a number of terms that refer to recombinant DNA technology. In order to provide a clear and consistent understanding of the invention, the following definitions are provided.

Cloning Vector

A plasmid or phage DNA or other DNA sequence which is able to replicate autonomously in a host cell and which is characterized by one or a small number of restriction endonuclease recognition sites. A foreign DNA fragment may be spliced into the vector at these sites in order to bring about the replication and cloning of the fragment. The vector may contain a marker suitable for use in the identification of transformed cells. For example, a marker may provide tetracycline resistance or ampicillin resistance.

Expression Vector

A vector similar to a cloning vector but which is capable of inducing the expression of the DNA that has been cloned into it after transformation into a host. The cloned DNA is usually placed under the control of (i.e., operably linked to) certain regulatory sequences such as promoters or enhancers. Promoters may be constitutive, inducible or repressible.

Recombinant Protein

A recombinant protein or recombinant receptor is a non-endogenous protein produced by the introduction of an expression vector into host cells. The term “non-endogenous” refers to any gene introduced into a cell by transfection. Thus, the transfection of a gene into a host cell for the purpose of producing large amounts of recombinant protein would constitute recombinant expression even though the gene might also be naturally present in the host cell.

Host

Any prokaryotic or eukaryotic cell that is the recipient of an expression vector or cloning vector is the “host” for that vector. Examples of cells that can serve as hosts are well known in the art, as are techniques for cellular transformation (see, e.g., Sambrook, et al., Molecular Cloning: A Laboratory Manual, 2^{nd} ed., Cold Spring Harbor (1989)).

Promoter

A DNA sequence typically found in the 5′ region of a gene, located proximal to the start codon. Transcription is initiated at the promoter. If the promoter is of the inducible type, then the rate of transcription increases in response to an inducing agent.

Expression

Expression is the process by which a polypeptide is produced from DNA. The process involves the transcription of the gene into mRNA and the subsequent translation of this mRNA into a polypeptide.

### DETAILED DESCRIPTION OF THE INVENTION

The present invention is directed to assays that can be used for identifying compounds that modulate the binding of sphingosine-1 PO₄ to the RP-23 receptor and which thereby alter the biological consequences of this interaction. The sphingosine-1 -PO₄ used as a ligand in assays may be obtained commercially (Sigma, St. Louis, Mo.) or can be synthesized using standard methodology well known in the art. It may be detectably labeled with radioisotopes such as ³²P, with fluorescent labels or with chemiluminescent labels. Alternatively, the ligand may be linked to enzymes, e.g., horseradish peroxidase, that are readily detectable in ELISA type procedures.

The RP-23 receptor for assays may be obtained from a variety of sources. For example, tissues or cells known to produce a large amount of the receptor may be used or, alternatively, assays may employ cells that have been cloned to express large amounts of receptor. The invention is limited to RP-23 genes and proteins whose structures have been described in the art in sufficient detail to distinguish them from all other G protein-coupled receptors and for which isolation procedures are known. This would include receptors from the human and mouse. For example, RP-23 may be cloned from murine T-lymphocytes using the procedure described by Harrigan, et al., (Molecular Endocrinol. 5:1331-1338 (1991)) or from human cells using procedures described in EP 789076. Alternatively, the gene sequence may be obtained by PCR using primers based upon the published sequences.

Once obtained, the RP-23 gene sequence should be incorporated into an expression vector with a promoter, preferably a promoter active in mammalian cells (see, Sambrook, et al., Molecular Cloning: A Laboratory Manual, 2^{nd} ed., Cold Spring Harbor Press (1989)). Examples of promoters that may be used include that of the mouse metallothionein I gene (Hamer, et al., J. Mol. Appl. Gen. 1:273-288 (1982)), the immediate-early and TK promoter of herpes virus (Yao, et al., J. Virol. 69:6249-6258 (1995); McKnight, Cell 31:355-365 (1982)); the SV 40 early promoter (Benoist, et al., Nature 290:304-310 (1981)); and the CMV promoter (Boshart, et al., Cell 41:521-530 (1985)). Vectors may also include enhancers and other regulatory elements.

As an alternative to conventional expression vectors, vectors for homologous recombination may be used to either incorporate a RP-23 gene into a host genome or to position a strong promoter at a location where it enhances the expression of an endogenous RP-23 gene. For the purposes herein, vectors designed for homologous recombination are also considered to be “expression vectors.”

Once expression vectors have been constructed, they can be introduced into a mammalian cell line by any method known in the art. This includes calcium phosphate precipitation, microinjection, electroporation, liposomal transfer, viral transfer. or particle mediated gene transfer. Although other mammalian cells may be used, HEK-293 cells have been found to give successful results and a procedure for expressing RP-23 in these cells is described in the Examples section. Standard procedures for selecting cells and for assaying for the expression of RP-23 (e.g. by Northern analysis) may be performed.

Once sphingosine-1 PO₄ and cells producing RP-23 have been obtained, assays may be performed to determine whether test compounds have an effect on binding and subsequent receptor-mediated events. A wide variety of assays suitable for these purposes are known in the art. For example, in radioligand binding assays, cells expressing RP-23 are incubated with sphingosine-1-PO₄ and with a compound being tested for binding activity. The preferred source of RP-23 is recombinantly transformed HEK-293 cells. Other cells may also be used provided they do not express proteins other than RP-23 that strongly bind sphingosine-1 PO₄. This can easily be determined by performing binding assays on cells transformed with an expression vector encoding RP-23 and comparing the results obtained with those obtained using their untransformed counterparts.

Assays may be performed using either intact cells or with membranes prepared from cells (see, e.g., Wang, et al., Proc. Natl. Acad. Sci. USA 90:10230-10234 (1993)). Either the membranes or cells are incubated with sphingosine-1-PO₄ and with a preparation of the compound being tested. After binding is complete, receptor is separated from the solution containing ligand and test compound e.g., by filtration, and the amount of binding that has occurred is determined. Preferably the ligand, i.e., sphingosine-1 PO₄, is detectably labeled with a radioisotope such as ³²P. However, other types of labels are also compatible with the invention. Among the most commonly used fluorescent labeling compounds are fluorescein, isothiocynate rhodamine, phycoerythrin, phycocyanin, allophycocyanin o-phthaldehyde and fluorescamine. Useful chemiluminescent compounds include luminol, isoluminol, theromatic acridinium ester, imidazole, acridinium salt, and oxalate ester.

Nonspecific binding may be determined by carrying out the binding reaction in the presence of a large excess of unlabeled ligand. For example, labeled sphingosine-1-PO₄ may be incubated with receptor and test compound in the presence of a thousand fold excess of unlabeled sphingosine-1-PO₄. Nonspecific binding should be subtracted from total binding, i.e., binding in the absence of a large excess unlabeled ligand, to arrive at the specific binding for each sample tested. Other steps such as washing, stirring, shaking, filtering and the like, may be included in the assays as necessary. Typically, wash steps are included after the separation of membrane-bound ligand from ligand remaining in solution and prior to the quantitation of the amount of ligand bound, e.g., by counting radioactive isotope. The specific binding obtained in the presence of test compound is compared with that obtained in the presence of labeled ligand alone to determine the extent to which the test compound has displaced receptor binding

In performing assays, care must be taken to avoid artifacts which may make it appear that a test compound is interacting with receptor when, in fact, binding is being inhibited by some other mechanism. For example, the compound being tested should be in a buffer which does not itself substantially inhibit the binding of sphingosine-1-PO₄ and should, preferably, be tested at several different concentrations. Preparations of test compound should also be examined for proteolytic activity and it is desirable that proteases be included in assays. Finally, it is highly desirable that the compounds identified as displacing the binding of sphingosine-1-PO⁴ to RP-23 receptor be examined in a concentration range sufficient to perform a Scatchard analysis on the results. This type of analysis is well known in the art and can be used for determining the affinity of the test compound for RP-23 receptor (see, e.g., Ausubel, et al., Current Protocols in Molecular Biology, 11.2.1-11.2.19 (1993); Laboratory Techniques in Biochemistry and Molecular Biology, Work, et al., ed., N.Y. (1978) etc.). Computer programs may be used to help in the analysis of results (see, e.g. Munson, Methods Eznymol. 92:543-577 (1983); McPherson, Kinetic, EBDA Ligand, Lowry—A Collection of Radioligand Binding Analysis Programs, Elsevier-Biosoft, E. K. (1985)).

Depending upon their effect on receptor activity, agents that inhibit the binding of sphingosine-1- PO₄ to RP-23 may either be agonists or antagonists. Activation of receptor may be monitored using a number of different methods. For example, adenylyl cyclase assays may be performed by growing cells in the wells of a microtiter plate and then incubating the wells in the presence or absence of test compound. cAMP can then be extracted in ethanol, lyophilized, and resuspended in assay buffer. Assay of cAMP thus recovered may be carried out using any method for determining cAMP concentration, e.g., the Biotrack cAMP Enzyme-immunoassay System™ (Amersham) or the Cyclic AMP [³H] Assay System (Amersham). Typically, adenylyl cyclase assays will be performed separately from binding assays, but it may also be possible to perform both assays on a single preparation of cells.

Activation of receptor may also be determined based upon a measurement of intracellular calcium concentration. For example, transformed HEK-293 cells may be grown on a glass cover slide or 96-well plate to confluence. After rinsing, the cells may be incubated in the presence of an agent such as Fluo-3 or FURA-2 AM (Molecular Probe F-1221). After further rinsing and incubation, calcium displacement may be measured using a photometer or a FLIPR (fluorescent imaging plate reader). Other types of assays for determining intracellular calcium concentrations are well known in the art and may also be employed. Assays that measure intrinsic activity of the receptor, such as those based upon inositol phosphate measurement, may be used in order to determine the activity of inverse agonists. Unlike antagonists, which block the activity of agonists but produce no activity of their own, inverse agonists produce a biological response diametrically opposed to the response produced by an agonist. For example, if an agonist promoted an increase in intracellular calcium, an inverse agonist would decrease intracellular at calcium levels.

In cases where RP-23 is provided by recombinant expression in a cell, it will usually be necessary to also recombinantly express an appropriate signal transducing G protein in the same cell in order to obtain receptors that not only bind ligand but that also stimulate adenylyl cyclase activity and calcium influx. The preferred G protein (to stimulate phospholipase C) for this purpose is Gαqi5, the gene for which may be obtained as described by (Conklin, B. R. et al., Nature 363, 274-276(1993). This gene may be inserted into an expression vector and used to transfect the cells transformed with the DNA encoding RP-23.

It is also possible to perform assays designed to identify modulators of sphingosine-1-PO₄ activity without using the ligand itself. These assays are based upon the observation that G protein-coupled receptors can be put into an active state even in the absence of their cognate ligand by expressing them at very high concentration in a heterologous system. For example, the receptor may be overexpressed using the baculovirus infection of insect Sf9 cells or the R-23 gene may be operably linked to a CMV promoter and expressed in COS or HEK-293 cells. In this activated state, antagonists of the receptor can be identified by measuring the ability of a test compound to inhibit constitutive cell signaling activity, e.g., by measuring adenylyl cyclase activity or changes in intracellular calcium concentration. For example, the intracellular concentration of calcium in the presence of test compound may be compared with the intracellular concentration in the activated cells alone. A statistically significant decrease in calcium levels in response to the test compound would be an indication that it is acting as an antagonist.

The assays described above merely provide examples of the types of assays that can be used for determining whether a particular test compound alters the binding of sphingosine-1 PO₄ to the RP-23 receptor and acts as an agonist or antagonist. There are many variations on these assays that are compatible with the present invention. Such assays may involve the use of labeled antibodies as a means for detecting sphingosine-1 PO₄ that is bound to receptor or may take the form of the fluorescent plate reader assays described in the Examples section below.

### EXAMPLES

I. Methods

The RP-23 gene (Harrigan et al., Molecular Endocrinol. 5:1331-1338 (1991)) was obtained and used to generate a mammalian expression vector. Specifically, 40 μg of RP-23 receptor DNA was digested with 100 units of EcoRI enzyme (Pharmacia) (Lambda ZAP II) at 37 degrees C., isolated on a 1% agarose gel and subcloned into the Eco RI site of pcDNA 3.0 (InVitrogen, San Diego, Calif.). The expression vector so produced was called pcDNA 3.0-RP-23. Plasmid DNA was prepared using the Qiaprep system from Qiagen.

Expression

HEK-293 cells were transfected with pcDNA 3.0-RP-23 using the calcium-phosphate method and subsequently transfected with DNA encoding a chimeric G protein (Gαqi5). This is Gq alpha with the C-terminal amino acids changed from EYNLV to DCGLF. The entire protein was subcloned in an expression vector (pCEP, Molecular Devices). A stable receptor pool of RP-23 and Gαqi5 was selected by applying selection agents (G418, 0.7 mg/ml and hygromycin 0.35 μg/ml) and maintaining cells in selection medium. The presence of mRNA specific for clone RP-23 was assessed by Northern Blot Analysis and by the reverse transcriptase polymerase chain reaction (RT-PCR).

Ligands

In order to identify the ligand of the RP-23 receptor, a collection of compounds was obtained from commercial sources (Sigma, CalBiochem, American Peptide Company, Bachem, RBI). The compounds were dissolved in water/DMSO at 3 μM and placed in 96 well microplates. A total of 846 compounds (peptides and non-peptides) were prepared and tested.

Assay

A functional assay was performed with FLIPR using the fluorescent calcium indicator dye Fluo-3 (Molecular Probes) on a 96 well platform. HEK-293 cells, either expressing the receptor with the chimeric G protein (Gαqi5) or wild type cells were loaded with Fluo-3 as follows. Stable HEK-293 clones expressing RP-23 receptor with Gαqi5and/or cells expressing RP-23R alone, or parental cells were plated at a density of 70,000 cells/well in a 96 well plate. On the day of the experiment, the RP-23 receptor expressing cells were loaded with fluorescent solution (Dulbecco's modified medium with 10% fetal bovine serum containing 4 μM Fluo-3 and 20% pluronic acid). The cells were incubated at 37° C. for one hour in a humidified chamber. Following the incubation, cells were Washed five times in Hanks' with 20 mM Hepes and 0.1% BSA (pH 7.4). The cells were analyzed using the FLIPR system to measure the mobilization of intracellular calcium in response to different compounds.

II. Results

HEK-293 cells that endogenously express some GPCRs such as PACAP receptors can be used as an internal control for assays. Background signal was established with all of the compounds in the parental HEK-293 cells (non-transfected) or parental HEK-293 cells transfected with Gαqi5 chimeric protein using the FLIPR assay. HEK-293 cells expressing the RP-23 receptor together with Gαqi5 were stimulated with all compounds and calcium responses were compared with those, in parental HEK-293 cells and HEK-293 cells transfected with Gαqi5. One compound, sphingosine-1-PO₄, consistently elicited a significant signal in the transfected cells expressing the RP-23 receptor and Gαqi5 chimeric protein as compared to the control cells. This indicates that sphingosine-1-PO₄ is interacting with the recombinantly expressed receptor. Confirmation of this conclusion was obtained by the observation of a dose-response relationship between sphingosine-1 PO₄ and the cells transfected with RP-23 and Gαqi5but not in non-transfected cells or in cells transfected with other receptors or Gαqi5 alone. Thus, it has been established that RP-23 is a specific receptor for sphingosine-1 PO₄ and that this receptor can be used to screen compounds which either mimic the action of sphingosine-1 PO₄ (agonists) or antagonize the action of sphingosine-1 PO₄ (antagonists).

Screening assays can be performed using thc FLIPR assay described above Alternatively, sphingosine-1 PO₄ can be phosphorylated with ³²P or tritiated and used as a tracer in radioligand binding assays on whole cells or membranes. Other assays that can be used include the GTPγS assay, adenylyl cyclase assays, assays measuring inositol phosphates and reporter gene assays (e.g., those utilizing luciferase, aqueorin, alkaline phosphatase, etc.).

All references cited herein are fully incorporated by reference. Having now fully described the invention, it will be understood by those of skill in the art that it may be performed within a wide and equivalent range of conditions, parameters and the like, without affecting the spirit or scope of the invention or any embodiment thereof.

What is claimed:

1. A method for determining whether a test compound modulates the binding of sphingosine-1-PO₄ to an RP-23 receptor, comprising:
a) incubating a source of RP-23 receptor with:
i) sphingosine-1-PO₄; and
ii) test compound,
b) determining the extent to which said sphingosine-1-PO₄ binds to said RP-23 receptor as the result of the incubation of step a);
c) determining the extent to which sphingosine-1-PO₄ binds to RP-23 receptor in control incubations which are performed under essentially the same conditions as the incubations of step a) but in which said test compound is absent or present at a significantly different concentration; and
d) concluding that said test compound modulates sphingosine-1-PO₄ binding if the amount of binding observed in step b) is either higher or lower than the binding observed in step c) to a statistically significant degree.

2. The method of claim 1, wherein said RP-23 receptor has the amino acid sequence of human RP-23.

3. The method of claim 1, wherein said source of RP-23 receptor is a cell expressing an RP-23 gene.

4. The method of claim 3, wherein said cell expresses a recombinant RP-23 gene.

5. The method of claim 3, further comprising determining whether said test compound significantly increases or decreases either the adenylyl cyclase activity of said cell or the intracellular calcium concentration of said cell.

6. The method of claim 1, wherein said sphingosine-1-PO₄ used in said incubations is detectably labeled.

7. The method of claim 1, wherein repeated incubations are performed in which the concentrations of RP-23 receptor and sphingosine-1-PO₄ are held constant and the concentration of test compound is varied.

8. The method of claim 1, wherein said control incubations are performed in the absence of said test compound.

9. A method for determining the ability of a test compound to modulate sphingosine-1-PO₄-stimulated RP-23 receptor-mediated activity comprising:
a) incubating a cell expressing RP-23 receptor with:
i) sphingosine-1-PO₄; and
ii) said test compound;
b) determining either the intracellular adenylyl cyclase activity or intracellular calcium concentration resulting from the incubation of step a);
c) determining the intracellular adenylyl cyclase activity or intracellular calcium concentration resulting from a control incubation performed under essentially the same conditions as the incubation of step a) but in which said test compound is absent or present at a significantly different concentration;
d) concluding that said test compound modulates sphingosine-1-PO₄-stimulated RP-23 receptor-mediated activity if the activity observed in step b) is either higher or lower than the activity observed in step c) to a statistically significant degree.

10. The method of claim 9, wherein said cell expresses a recombinant RP-23 gene.

11. The method of claim 9, wherein said RP-23 receptor has the amino acid sequence of human RP-23.

12. The method of claim 9, wherein said sphingosine-1-PO₄ is detectably labeled.

13. The method of claim 9, wherein repeated incubations are performed in which the concentration of receptor and sphingosine-1-PO₄ are held constant and the concentration of said test compound is varied.

14. The method of claim 9, wherein said control incubations are performed in the absence of said test compound.

15. A method of determining if a test compound is an antagonist of sphingosine-1-PO₄ RP-23 receptor activation, comprising:
a) incorporating a DNA molecule encoding said RP-23 receptor into an expression vector so that it is operably linked to a promoter;
b) transfecting the expression vector formed in step a) into a host cell;
c) selecting cells transfected in step b) that have constitutively activated RP-23 receptors as evidenced by a statistically significant increase in intracellular adenylyl cyclase activity or intracellular calcium concentration;
d) contacting the cells of step c) with said test compound, and
e) determining if said test compound causes a statistically significant decrease in either intracellular adenylyl cyclase activity or intracellular calcium relative to control cells not contacted with said test compound.

16. The method of claim 15, wherein said RP-23 receptor has the amino acid sequence of human RP-23.
